(19) United States
(12) Patent Application Publication
(10) Pub. No.: US 2010/0280403 A1
(43) Pub. Date: Nov. 4, 2010

(54) RAPID SERIAL PRESENTATION COMMUNICATION SYSTEMS AND METHODS

(75) Inventors: Deniz Erdogmus, Beaverton, OR (US); Brian Roark, Portland, OR (US); Melanie Fried-Oken, Portland, OR (US); Jan Van Santen, Lake Oswego, OR (US); Michael Pavel, Portland, OR (US)

Correspondence Address: Schwabe Williamson & Wyatt;PACWEST CENTER, SUITE 1900, 1211 SW FIFTH AVENUE, PORTLAND, OR, 97204, US

(73) Assignee: Oregon Health & Science University

(21) Appl. No.: 12/812,401

(22) PCT Filed: Jan. 12, 2009
(86) PCT No.: PCT/US09/30748

§ 371 Date: Jul. 9, 2010

Related U.S. Application Data
(60) Provisional application No. 61/020,672, filed on Jan. 11, 2008.

(51) Int. Cl. A61B 5/0482

(52) U.S. Cl. 600/545

(57) ABSTRACT

Embodiments of the disclosed technology provide reliable and fast communication of a human through a direct brain interface which detects the intent of the user. An embodiment of the disclosed technology comprises a system and method in which least one sequence of a plurality of stimuli is presented to an individual (using appropriate sensory modalities), and the time course of at least one measurable response to the sequence(s) is used to select at least one stimulus from the sequence(s). In an embodiment, the sequence(s) may be dynamically altered based on previously selected stimuli and/or on estimated probability distributions over the stimuli. In an embodiment, such dynamic alteration may be based on predictive models of appropriate sequence generation mechanisms, such as an adaptive or static sequence model.

### CROSS REFERENCE TO RELATED APPLICATIONS

The present application claims priority to U.S. Patent Application No. 61/020,672, filed Jan. 11, 2008, entitled “Rapid Serial Presentation Communication Systems and Methods,” the entire disclosure of which is hereby incorporated by reference in its entirety.

### TECHNICAL FIELD

Embodiments of the disclosed technology relate to the field of assistive communication devices, and, more specifically, to methods, apparatuses, and systems for enabling brain interface communication.

### BACKGROUND

A primary challenge in empowering people with severe speech and physical impairments (SSPI) to verbally express themselves through written and spoken language is to increase the communication rate. These difficulties are faced by people with SSPI, such as those with severe spinal cord or brain injury (resulting in locked-in syndrome (LIS)) or neuromuscular disease (such as Amyotrophic Lateral Sclerosis, ALS). As an example, LIS is a condition consisting of nearly complete paralysis due to brainstem trauma, which leaves the individual aware and cognitively active, yet without the ability to move or speak. These disabilities limit the utility of Augmentative and Alternative Communication (AAC) technologies relying on motor control and movement, even of the eyes. AAC technologies rely heavily on the accurate assessment of the intent of the user. In generating verbal communication (for both written and spoken output), a system must identify the intended symbol sequences, whether the symbols are letters, words, or phrases, and accurately classify the observed indicators (any relevant physical signal) into a set of possible categories. The system's pattern recognition performance is a crucial component for the overall success of the assistive technology.

For patients with LIS, for example, movement is typically limited to blinking and vertical eye movement, thus precluding the use of creative eye tracking text input systems. This situation currently limits users to the use of eye-blink technologies for text input, yielding excruciatingly slow communication rates of around 1 word per minute, particularly for unconstrained free text input, thus effectively precluding extensive interaction.

Similar difficulties are also seen with children with Autism Spectrum Disorders (ASD) as such individuals are heterogeneous in their verbal and nonverbal communication abilities, neurocognitive profiles, and motor abilities. Further, there is a subset of children with ASD who (i) lack expressive speech and language; (ii) are too dyspraxic to operate a keyboard, pointing device, or other typical AAC device; (iii) but who, despite appearances of possible mental retardation, are nevertheless assumed to have adequate cognition, literacy, and receptive language understanding.

Current systems are unable to provide adequate communication rates and methodologies for the types of individuals mentioned above.

### BRIEF DESCRIPTION OF THE DRAWINGS

Embodiments of the disclosed technology will be readily understood by the following detailed description in conjunction with the accompanying drawings. Embodiments of the technology are illustrated by way of example and not by way of limitation in the figures of the accompanying drawings.

FIG. 1 illustrates feature projections, in accordance with an embodiment of the disclosed technology, from 35 features obtained from the retained 7 EEG electrodes to n-dimensional hyperplanes revealing that classification accuracy on the validation data reaches a plateau. Linear projections proposed by ICA followed by selection based on validation error rate (ICA-Error) is best as expected (being a wrapper method), ICA followed by mutual information based selection (ICA-MI) is less effective but is better than projections offered by linear discriminant analysis (LDA).

FIG. 2 illustrates ROC curves of linear and nonlinear projections, in accordance with an embodiment of the disclosed technology, to a single dimension on the BCI Competition III dataset V. Methods are ICA-based MI estimate and feature selection, ICA projection with MI-based selection, LDA, Kernel LDA, and Mutual Information based nonlinear projection.

FIG. 3 illustrates a closed-loop interface system in accordance with an embodiment of the disclosed technology.

### DETAILED DESCRIPTION OF EMBODIMENTS OF THE DISCLOSED TECHNOLOGY

In the following detailed description, reference is made to the accompanying drawings which form a part hereof, and in which are shown by way of illustration embodiments in which the disclosed technology may be practiced. It is to be understood that other embodiments may be utilized and structural or logical changes may be made without departing from the scope of the disclosed technology. Therefore, the following detailed description is not to be taken in a limiting sense, and the scope of embodiments in accordance with the disclosed technology is defined by the appended claims and their equivalents.

Various operations may be described as multiple discrete operations in turn, in a manner that may be helpful in understanding embodiments of the disclosed technology; however, the order of description should not be construed to imply that these operations are order dependent.

The description may use perspective-based descriptions such as up/down, back/front, and top/bottom. Such descriptions are merely used to facilitate the discussion and are not intended to restrict the application of embodiments of the disclosed technology.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the phrases “in an embodiment,” or “in embodiments,” which may each refer to one or more of the same or different embodiments. Furthermore, the terms “comprising,” “including,” “having,” and the like, as used with respect to embodiments of the disclosed technology, are synonymous.

In various embodiments of the disclosed technology, methods, apparatuses, and systems for enabling brain interface communication are provided. In exemplary embodiments of the disclosed technology, a computing system may be endowed with one or more components of the disclosed apparatuses and/or systems and may be employed to perform one or more methods as disclosed herein.

Embodiments of the disclosed technology provide reliable and fast communication of a human through a direct brain interface which detects the intent of the user. Embodiments may enable persons, such as those with severe speech and physical impairments, to control a computer/machine system through verbal commands, write text, and communicate with other humans in face-to-face or remote situations. In an embodiment, healthy humans may also utilize the proposed interface for various purposes.

An embodiment of the disclosed technology comprises a system and method in which at least one sequence of a plurality of stimuli is presented to an individual (using appropriate sensory modalities), and the time course of at least one measurable response to the sequence(s) is used to select at least one stimulus from the sequence(s). In an embodiment, the sequence(s) may be dynamically altered based on previously selected stimuli and/or on estimated probability distributions over the stimuli. In an embodiment, such dynamic alteration may be based on predictive models of appropriate sequence generation mechanisms, such as an adaptive or static natural language model.

An embodiment of the disclosed technology may comprise one or more of the following components: (1) rapid serial presentation of stimuli, such as visual presentation of linguistic components (e.g., letters, words, phrases, and the like) or non-linguistic components (e.g., symbols, images, and the sort), or other modalities such as audible presentation of sounds, optionally with individual adjustment of presentation rates, (2) a user intent detection mechanism that employs multichannel electroencephalography (EEG), electromyography (EMG), evoked-response potentials (ERP), input buttons, and/or other suitable response detection mechanisms that may reliably indicate the intent of the user, and (3) a sequence model, such as a natural language model, with a capability for accurate predictions of upcoming stimuli that the user intends in order to control the upcoming sequence of stimuli presented to the subject.

In an embodiment, the speed of presentation may be adjusted as desired with the phrase “rapid” not restricting the speed in any way. In an embodiment, an intent detection algorithm may evaluate the measured input signal (e.g., EEG, EMG, ERP, or any combination of suitable neural, physiological, and/or behavioral input signals) and select the intended/desired stimulus from the sequence based on likelihood assignments. Future entries of the presentation sequence may be determined according to previous selections and/or determinations of intent based on the sequence model. The sequence model may also adapt over time, if desired, in order to reflect the style and preferences of the user. An embodiment may also incorporate a speech synthesis component to enable spoken communication. Further, an embodiment may also include an attention monitoring component to minimize misses and false detections of intent due to reduced attention of the subject or increased cognitive load.

Invasive and noninvasive brain interface techniques that are in existence today rely on presenting all possible options on a screen simultaneously (distributed spatially) and either predicting the user intent from among all possibilities, or allowing the user to control a cursor over the two dimensional keyboard layout (minimum of 4 choices per action with multiple correct decisions required to reach an intended letter). Alternatively, systems based on eye tracking or button/joystick/mouse inputs usually utilize predictive word completion components, but require close-to-healthy motor control of various muscles. Embodiments of the disclosed technology eliminate or alleviate the intent detection problem through a brain interface by distributing the options over time. In an embodiment, at each instant, a binary question, such as a “yes/no” type question, may be posed, or intent may be classified over multiple choices (nonbinary) indicating degrees of recognition or acceptability of a particular stimulus. In a binary “yes/no” type embodiment of the system, for example, the user only needs to focus on the temporal sequence and recognize the desired stimulus (such as a letter/word/phrase), thus implicitly generating a “yes” response when viewing the desired element. Further, for example, a “no” response may be implied by the user taking no action or showing no sign of recognition. In other embodiments, the response levels may be correlated to the level of acceptability of a particular stimulus or a level of agreement by the user with a particular presented stimulus. In embodiments, user convenience may be provided, intent detection difficulty may be managed, and with the introduction of predictive models for sequence generation and sequence preparation, speeds comparable to handwriting by an average general-population sample are made possible (such as tens of words per minute). Other brain interface methods for communication, on the other hand, measure speeds at letters per minute or slower metrics.

In an embodiment, a user may be presented with linguistic stimuli (e.g., letters) or non-linguistic stimuli (e.g., symbols, images, and the sort) at a single position on the interface, thus removing the need for eye movement or active cursor control. Other positions, individually or in combination, on an interface may be utilized in embodiments, as desired.

In an embodiment, evoked response potentials (ERP) may be captured through a noninvasive multichannel electroencephalogram (EEG) brain interface to classify each stimulus as the target or not. In an embodiment, of particular importance may be the P300 potential, which peaks at around 300 ms after the presentation of a stimulus that the user has targeted. Such effects may be exploited for fast localization of target stimuli with stimulus presentation durations of as little as 75 ms, thus illustrating that the latency of the neural signal being used for classification does not dictate the optimal latency of the presentation of the stimuli. For example, in accordance with an embodiment, if a user is presented with letters in alphabetic order, each for 100 ms, and the P300 is detected with a peak after 650 ms, then such detection would indicate that the target occurred at 350 ms, i.e., the letter “D”. The effort required of the user is to look for a particular linguistic stimulus, such as the next letter of the word they are trying to produce. In an embodiment, rich linguistic models may be used to order the symbols dynamically, in order to reduce the number of symbols required to reach the desired target. An explicit action of choosing is not required, since the neural signal indicating recognition of the target is an involuntary response and serves to select it from among the available choices. In embodiments, reducing the latency of symbol presentation and improving the quality of the predictive language models jointly increases communication rates within an interface requiring no movement at all.

In an embodiment of the disclosed technology, there is provided an EEG-based brain interface that enables the classification of “yes/no” intent in real-time through single-trial ERP detection using advanced signal processing, feature extraction, and pattern recognition algorithms. Embodiments may utilize information theoretic and Bayesian statistical techniques for robust dimensionality reduction (e.g., feature extraction, selection, projection, sensor selection, and the sort). Embodiments may also utilize hierarchical Bayesian modeling (e.g., mixed effect models) to model the temporal variations in the EEG signals, associated feature distributions, and decision-directed learning to achieve on-going optimization of session-specific ERP detector parameters. In addition, embodiments may provide a real-time classification system that accurately determines (i) the intent (e.g., binary intent) of the user (e.g., via ERPs), and (ii) the attention level of the user (e.g., via features extracted from the EEG).

In an embodiment of the disclosed technology, there is provided an optimal real-time, causal predictive, open-vocabulary, but context-dependent natural language model to generate efficient sequences of language components that minimize uncertainty in real-time intent detection. An embodiment provides accurate probabilistic large-vocabulary language models that minimize uncertainty of upcoming text and exhibit high predictive power, with sub-word features allowing for open-vocabulary use. In an embodiment, there are provided learning techniques integrated in the systems that allow perpetual, on-line adaptation of the language models to specific subjects based on previously input text. In addition, an embodiment provides optimal presentation sequence generation methods that help minimize uncertainty in intent detection and minimize the number of symbols presented per target.

While certain embodiments herein focus on single-trial ERP detection from EEG measurements, alternative modalities may involve electrocorticogram (ECoG) and/or local field potential (LPF) based ERP detectors for improved signal to noise ratios. In an embodiment, choice of the EEG paradigm allows for signal processing and pattern recognition techniques for this noninvasive brain interface modality that may be utilized for human-computer interaction applications that benefit the general population. In embodiments, methods described herein for EEG processing may also be applicable to the invasive signal acquisition alternatives without loss of generality.

An embodiment of the disclosed technology provides real-time causal predictive natural language processing. Predictive context-aware large and open vocabulary language models that adapt to the particular language style of the user may increase communication rates with the embodiments of the disclosed technology, particularly when allowing for unconstrained free text entry that may frequently include what would otherwise be out-of-vocabulary terms, such as proper names. Of particular interest is the use of large-vocabulary discriminative language modeling techniques, which may, in an embodiment, contribute constraints to the model without restricting out-of-vocabulary input. Differences between this language modeling paradigm and more typical language modeling applications, such as speech recognition and machine translation, lead to model adaptation scenarios that allow for on-line customization of the interface to the particular user.

In accordance with an embodiment of the disclosed technology, a rapid serial presentation device sequentially presents images of text units at multiple scales (e.g., letter/word/phrase) appropriately determined by a predictive language model and seeks confirmation from the user through brain activity by detecting ERP waveforms associated with positive intent in response to the visual recognition of the desired text unit. Existing techniques rely on presenting multiple options, some with rudimentary language models, to the user and aim to classify the intent among these multiple possibilities, all being highly likely candidates. Many of such techniques have interfaces that include various forms of button and mouse controls. Clearly, such interfaces are not suitable for patients who lack motor control and for those who present with neurodegenerative conditions that prevent highly accurate eye movement control. In an embodiment of the disclosed technology, the intent of the user is determined from brain activity, and thus such embodiments still benefit people with SSPI, including those who lack volitional motor control of eye movements. In an embodiment, alternative binary intent detection mechanisms such as a button press may also be employed. In addition, in an embodiment, the presentation rate may be adapted to match the pace of the user, and for persons who are challenged by time-critical tasks, it may, for example, be slowed down. In embodiments, exemplary presentation rates for each stimulus may be approximately 100 ms, 200 ms, 300 ms, 400 ms, 500 ms, or more.

Benefits of embodiments of the disclosed technology are provided, in part, by the fast sequential binary intent detection mechanism. Existing techniques rely on the classification of the intent of the user from a large number of possible options presented all at once. Since the potential uncertainty measured by the entropy of the prior class distributions increases with the number of possible classes, the pattern recognition problem becomes more difficult on average. Embodiments of the disclosed technology present a sequence of binary questions trying to detect a simple “yes” response. Distributing the multi-hypothesis problem over time to multiple binary hypothesis testing problems reduces the expected classification difficulty of each decision. As indicated above, nonbinary options for response may be acceptable as an alternative to, or in addition to, a binary response paradigm. In an embodiment, the difficulty may be further reduced with sequence model predictions by isolating likely candidates from each other with highly unlikely candidates.

Embodiments of the disclosed technology enable people with severe speech and motor impairments to interface with computer systems for the purpose of typing in order to establish and maintain seamless spontaneous communication with partners in face-to-face situations, as well as in remote environments such as Internet chat, email, or telephone (via text-to-speech). In addition, embodiments also enable the target population to access information available on the Internet through a computer. In an embodiment, user adaptability capabilities embedded through individualized language preference modeling also ensures seamless personal expression.

In embodiments, the temporal sequencing approach (1) eliminates the need to have consecutive correct interface decision sequences to get one stimulus selected (as opposed to the grid-layout or hierarchical designs), (2) does not require active participation and planning from the user in a struggle to control the interface apart from focusing attention to the presented sequence in search of the desired target, and (3) removes the challenge to determine the correct intent among a large number of concurrent possibilities (as opposed to designs where the user focuses on one of many possibilities).

In an embodiment, there is provided an EEG-based brain interface to detect the positive intent of the user in response to the desired image that has been presented in a rapid serial presentation mode. EEG data may be collected using the standard International 10/20 electrode configuration. In embodiments, eye and other artifacts (if present) may be measured by appropriate reference electrodes and may be eliminated in the preprocessing stage using adaptive filtering techniques. In addition, statistical models are provided to discriminate negative and positive intent (i.e., detect ERP in background neural activity). In an embodiment, dimensionality reduction methods may be provided to maximally preserve discriminative information (e.g., a method that identifies relevant features & eliminates irrelevant features).

In embodiments, EEG-based brain interfaces may utilize one or more relatively standard features for intent or cognitive state classification, such as: (1) temporal signals from channels concatenated to a large vector (e.g., at 256 Hz, ERP detection using time-aligned 1 s-long post-stimulus windows from 32 channels would yield 256×32=8192 dimensional feature vectors); (2) power spectral density based features, typically in the form of AR-modeling (e.g., equivalent to linear prediction coefficients in speech) or a windowed estimate averaged over established clinically relevant bands (e.g., alpha, beta, theta, etc; for five frequency bands or five AR coefficients and 32 channels, where one would have 160 dimensional features and although clinical bands are common and provide reasonably good results, they are usually suboptimal for brain interface design purposes); (3) wavelet decomposition based features (typically a discrete wavelet transform causes an explosion in feature dimensionality—at 256 Hz 1 s-long post stimulus signal length with 32 channels, one would obtain 32×256×l features for an l-level wavelet analysis and then use some dimensionality reduction technique such as “best basis selection” to reduce dimensionality). In addition, in embodiments, phase synchronization in EEG and fractal and chaotic nature of EEG signals are properties that may be exploited for extracting features for brain interface design. Thus, in an embodiment, fractal and 1/f-process based models of EEG signals may be utilized to extract alternative discriminative features.

In embodiments, one or more of temporal, spectral, wavelet, and fractal properties of the EEG signals may be used to detect positive intent of the subjects. The utilization of a large number of sources in pattern recognition tasks is in principle beneficial, since discriminative information contained in a set of features is at least equal to that contained in any subset or low-dimensional projection. In practice, however, pattern recognition systems are trained with a finite amount of data, thus issues of generalization may utilize dimensionality reduction in order to maintain significantly informative dimensions through feature projections (e.g., including selection).

Qualitatively, in accordance with an embodiment of the disclosed technology, the goal of dimensionality reduction is to identify a submanifold embedded in the Euclidean feature space on which the probability distributions of the classes of interest background and ERP for the rapid serial presentation device are projected. Given a high dimensional feature vector xε [symbol] ⁿ the aim is to determine a smooth mapping f: [symbol] ⁿ→ [symbol] ^{m} where m<n such that the reduced dimensional feature vector is y=f(x). There are two components to identifying such an optimal manifold: projection topology (e.g., linear/nonlinear) with various constraints, and discriminability measure. The topology determines the structural constraints placed on the dimensionality reduction scheme. In general, nonlinear projections are sought, but since such projections may be learned from data using statistical techniques, generalization and overfitting concerns need to be properly addressed. Clearly, linear projections of the form y=Wx, where Wε [symbol] ^{n×m}, are a convenient subset that are structurally constrained to Euclidean subspaces, thus are severely regularized projection models. Further, in an embodiment, one could limit the projection matrix to consist of only one nonzero entry per row, and thus obtain a feature selection scheme.

In an embodiment, while nonlinear and linear projections are more capable of preserving discriminative information, feature subset selection is a particularly relevant procedure when one is selecting a subset of sensors (e.g., EEG electrodes) in order to manage hardware system complexity without significantly losing performance. In the context of brain interfaces, EEG channel selection may be used to simplify overall system design by reducing communication bandwidth (e.g., less data to be transmitted/recorded) requirements and real-time computational complexity (e.g., less data to be processed). In the context of application to subjects with LIS, simplifying the system is also beneficial to minimize cable and electronic clutter since the subjects are generally already surrounded by numerous monitoring and life-support devices.

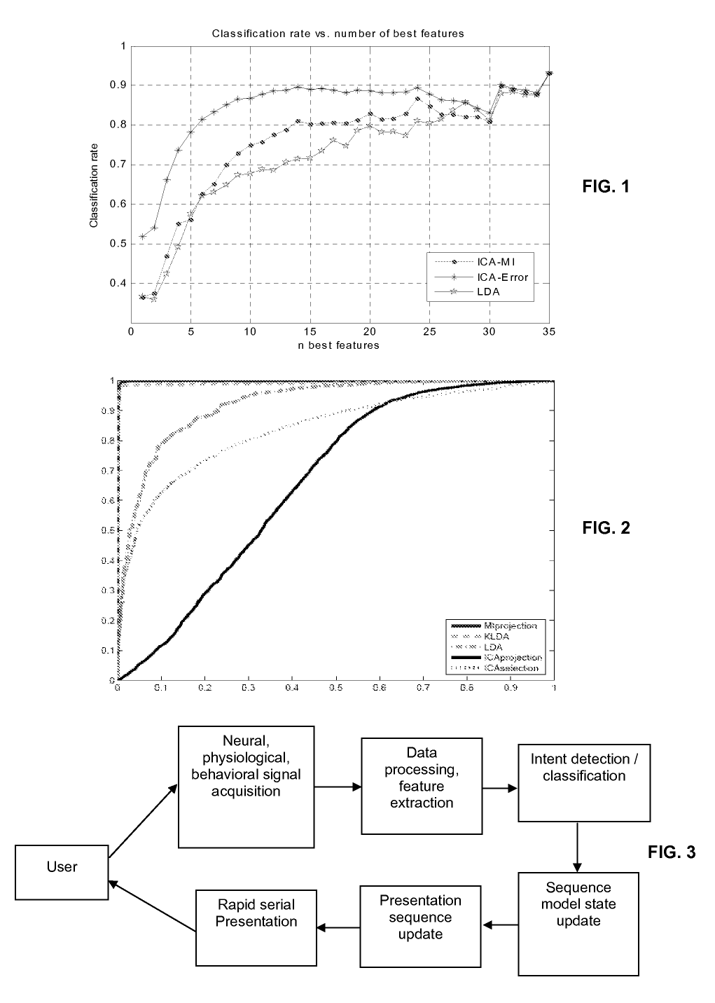

In embodiments, cognitive state estimation linear projections may be used to address certain computational constraints. An embodiment may utilize the maximum mutual information principle to design optimal linear projections of high dimensional PSD-features of EEG into lower dimensions. For example, FIG. 1 illustrates a projection of 35 dimensional features (5 clinical PSD bands from 7 selected EEG channels) to lower dimensional hyperplanes using independent component analysis followed by projection selection based on classifier error and mutual information on the validation set for a two-class problem. Information theoretic measures such as the optimality criterion in Equation 1 using Renyi's entropy may be regarded as a generalization of the concept behind Fisher's discriminant and may be utilized to determine linear or parametric nonlinear projections:

J_{α}(W)=H_{α}(Wx)−Σ_{c}p_{c}H_{α}(Wx|c) Equation 1

An embodiment of the disclosed technology demonstrates a comparison of linear and (nonparametric) nonlinear projection methods for the BCI Competition III dataset V containing data from 3 subjects in 4 nonfeedback sessions. From the raw 32-channel EEG data (512 Hz sampling rate), the EEG potentials were filtered by surface Laplacians, then a PSD estimate in the band 8-30 Hz was obtained for 1 s windows starting every 62.5 ms with a frequency resolution of 2 Hz. Using the 8 centro-parietal channels (C3, Cz, C4, CP1, CP2, P3, Pz, P4), a 96-dimensional feature vector was obtained. Five-fold cross-validation ROC curve estimates of 1-dimensional projections to classify imagined right/left hand movements are presented in FIG. 2. Imagined movements may be used to create intent-communicating brain signals in some embodiments of the proposed interface.

In embodiments, feature selection and projections may be used to identify and rank relevant EEG channels toward achieving optimal performance with minimal hardware requirements. A particular embodiment provides for the formation of pruned graphical conditional dependency models between features to reduce the effects of curse of dimensionality in optimal feature projection and selection. As an example, the use of pairwise dependencies between features may be utilized to form an affinity matrix and to determine subspaces of independent features. Other higher-dimensional dependencies may be utilized to reduce the dimensionality of the joint statistical distributions that may be estimated from finite amounts of data. In accordance with embodiments, such features benefit the general application area of EEG-based brain computer interfaces by providing principled and advanced methodologies and algorithms for feature extraction from EEG signals for classification of mental activity and intent. More broadly, such features benefit the field of data dimensionality reduction for pattern recognition and visualization.

In an embodiment, the single-trial ERP detection problem is essentially a statistical hypothesis testing question where the decision is made between the presence (class label 1, the null hypothesis) and nonpresence (class label 0, the alternative hypothesis) of a particular signal in measurements corrupted by background EEG activity and noise. In an embodiment, given the features based on which the statistical evidence may be evaluated, an optimal decision that minimizes the average risk may be given by a Bayes classifier. Let xε [symbol] ⁿ be the feature vector to be classified and denote the class-conditional distributions by p(x|c) where cε{0,1}. Also denote class prior probabilities with p_{c} and risk associated with making an error in classifying class c, i.e., P(decide not-c|truth is c), with r_{c}. Then, the average risk is p₀r₀P(1|0)+p₁r₁P(0|1) and the optimal decision strategy, given the true class distributions (or estimates obtained from training samples), is the likelihood ratio test:

$$L ( x ) = \frac{p ( x \vert 1 )}{p ( x \vert 0 )} \begin{matrix}{>}^{Decide 1} \\ {<}_{Decide 0}\end{matrix} \frac{{r}_{0} {p}_{0}}{{r}_{1} {p}_{1}} \qquad Equation 2$$

In an embodiment, the Bayes classifier may be explicitly implemented using a suitable estimate of the class-conditional distributions from training data; parametric and nonparametric density models including the logistic classifier, standard parametric families possibly in conjunction with the simplifying naïve Bayes classifier methodology which assumes independent features), mixture models, and kernel density estimation or other nearest-neighbor methods. High dimensional features with relatively little training datasets create generalization and overfitting concerns that may be addressed with these density models. An alternative embodiment of an approach to the classification problem is to provide an implicit model by training a suitable classifier topology to learn the optimal decision boundary between the two classes; neural networks, support vector machines, and their variations are among the most popular approaches currently known. These classifiers also suffer from the curse of dimensionality, and neural network classifier models generally need to be regularized through cross-validation and other means; however, support vector machines inherently employ regularization in their construction and training. Training data may be obtained by instructing the subject to type notice a sequence of known target images (letters/words) such that sufficiently reliable ERP characterization (validated on novel targets) is possible.

Given the scarcity of training data in a typical brain interface application, embodiments of the disclosed technology provide techniques that (i) utilize regularization for classifier parameters in the form of prior distributions for parameters, and (ii) use on-line (real-time or other than real-time) learning techniques, both of which lead to a continuously adapting, subject-specific classifier. Hierarchical Bayesian approaches, specifically mixed effects modeling techniques, may provide the main utility in achieving regularization. For sake of illustration, consider a linear logistic classifier design. Suppose a given training set from one or more subjects across multiple sessions: {(x_{j,i},c_{j,i}),i=1, . . . , N^{j}}, where x are extracted reduced-dimensionality features from stimulus-aligned multi-channel EEG recordings and c are class labels for the corresponding stimuli. The linear-logistic classifier output is in the form y=f(x^{T}w), where f(a)=(1+e^{-a})⁻¹ (alternative monotonic nonlinearities with unit-range correspond to other class conditional cumulative density assumptions). In the mixed effects framework, data is pooled from multiple sessions, j=1, . . . , J, and the classifier weights are split to two terms representing the average and the session variability effects: yⱼ=f(x^{T}w+x^{T}uⱼ). A suitable prior for uⱼ needs to satisfy E[x^{T}uⱼ]=0 so that the session average of the classifiers reduce to w; typically as in variational Bayes techniques, E[uⱼ]=0 suffices and simplifies calculations. Assuming a zero-mean Gaussian with covariance D as the prior of uⱼ for illustration, the maximum likelihood model optimization problem is expressed as:

$${max}_{w , \{ {u}_{j} \} , D} {\sum }_{j} [ {\sum }_{{i}_{j}} log f ( {x}_{j , {i}_{j}}^{T} ( w + {u}_{j} ) ) + log N ( {u}_{j} ; 0 , D ) ] \qquad Equation 3$$

Maximization may be done via standard optimization techniques such as EM. Upon obtaining the optimal solution, D may be stored and for new training sessions it may be employed as prior knowledge to regularize the model and reduce training sample size requirements (therefore calibration time).

In an embodiment, on-line learning procedures may follow techniques utilized in decision-directed adaptation (commonly used in adaptive channel equalizers of communication channels) or semi-supervised learning (commonly utilized to exploit unlabeled data). Without significant risk, the preceding decisions of the ERP detector may be assumed correct if the user does not take corrective action to the text written by the rapid serial presentation device. Hence, a continuous supply of additional (probabilistically) labeled training data may be obtained during actual system usage. This data may be utilized to periodically adjust the classifier and/or the ERP/background models to improve performance and/or track drifting signal distributions due to the nonstationary nature of background neural activity, thus improving ERP detection accuracy. Even data with uncertain labels (which may occur due to various reasons such as temporary loss of attention) may be employed for further training using techniques similar to semi-supervised learning.

In an embodiment, another provided component relates to the exchange and utilization of decision confidence levels between the ERP detector and the language predictor. Optimal fusion of decisions made by the two components of the interface in the context of their estimated self-confidence levels ensures increased overall performance. For example, a potential missed ERP (e.g., indicated by a no-ERP decision with high associated uncertainty) may be taken into account by the language model in generating the next sequence element (e.g., perhaps re-present the symbol if it had high likelihood with high certainty).

In an embodiment, another factor to address for successful employment of the rapid serial presentation paradigm is monitoring of the subject's attention status in order to prevent misses due to low attention and/or cognitive overload due to speed. Consequently, in an embodiment, it is beneficial to obtain continuous real-time estimates of the attention and cognitive load levels of the subject from EEG measurements simultaneously with ERP detection. Estimates of attention level may be based on frequency distribution of power, interelectrode correlations, and other cross-statistical measures between channels and frequency bands.

In embodiments, suitable language modeling is needed to ensure rapid and accurate presentation of probable sequences. In applications that produce natural language sequences, such as Automatic Speech Recognition (ASR) and Machine Translation (MT), which produce target language sequences corresponding to speech or source language sequences, language models serve as a prior model for disambiguating between likely sequences and other system outputs. Standard n-gram models decompose the joint sequence probability into the product of smoothed conditional probabilities, under a Markov assumption so that the estimation, storage, and use of the models are tractable for large vocabularies. For a given string of k words w₁, . . . , wₖ, a trigram model gives the following probability

P(w₁ . . . wₖ)=P(w₁)P(w₂|w₁)Πᵢ₌₃^{k}P(wᵢ|wᵢ₋ₗwᵢ₋₂) Equation 4

where each conditional probability of a word given the input history may be smoothed using one of a number of well-known techniques. Log-linear models may also be used for estimating conditional and joint models for this problem.

In an embodiment, rapid serial visual presentation (RSVP) text entry also has a role for stochastic language models: given a history of what has been input, along with other contextual information, a determination may be made as to the most likely next words or symbols that the user may want to input. This use of language models differs from the typical use as presented above in several key ways. First, the embodiment does not have a use for the full joint probability of the input sequence; since the user may edit the input, each conditional distribution is used independently of the others. Second, under the assumption that the string input by the user is the intended string, the use of the system actually provides a stream of supervised training data, which may be used for system adaptation to a particular user. For example, novel proper names and frequently used phrases become easier to input as they are incorporated into the model. Finally, in an embodiment, in order to allow subjects to enter unconstrained text, the vocabulary of the model may not be fixed when it is used by the interface.

As an example, in accordance with an embodiment, consider how a language model may be utilized for this task. Let c denote the context of the input, which may include the history of what has been input previously as well as other contextual information that might be of use, such as the words in the message to which the user is replying. Let nεΣ denote the next symbol from the vocabulary Σ, which may be letters, sub-word sequences of letters, words, or even phrases. The probability of n given c may be defined via the dot product of a feature vector Φ derived from c and n, and a parameter vector α as follows:

$$P ( n \vert c ) = \frac{exp ( α \cdot Φ ( c , n ) )}{{\sum }_{m ∈ \sum } exp ( α \cdot Φ ( c , m ) )} \qquad Equation 5$$

Such models are known as log-linear models, since the log probability is simply the dot product of the feature and parameter vectors minus a normalizing constant. The estimation of conditional log-linear sequence models for ASR and MT may be done with iterative estimation techniques that are guaranteed to converge to the optimal solution, since the problem is convex. In embodiments, it may be preferable to use a global normalization rather than normalizing with the local context c, due to the label bias problem. In an embodiment, a user may correct mistaken predictions, so the context may be taken as “true”, which avoids such issues. As a result, these distributions may be normalized according to the local context, which greatly simplifies the estimation.

In an embodiment, the objective function of training is regularized conditional log-likelihood (LL_{R}), where the regularization controls for overtraining. Let N be the number of training examples, cᵢ the context of example i, and nᵢ the correct next symbol of example i. Then LL_{R} for a given parameter vector α is:

LL_{R}(α)=Σᵢ₌₁^{N}[α·Φ(cᵢ,nᵢ)−log Z(cᵢ,α)]−∥α∥²/(2σ²) Equation 6

where Z(cᵢ,α) is the appropriate normalization factor and σ² is the variance of the zero-mean Gaussian prior over the parameters. The value of σ dictates how much penalty is incurred for moving away from a zero parameter value, and is typically optimized on held out data. This function is convex; hence general numerical optimization routines making use of the local gradient may be used to estimate the model parameters. For parameter s, the gradient is:

$$\frac{\partial {LL}_{R} ( α )}{\partial {α}_{s}} = {\sum }_{i = 1}^{N} [ \begin{matrix}{Φ}_{s} ( {c}_{i} , {n}_{i} ) - \\ {\sum }_{m ∈ \sum } {p}_{α} ( m \vert {c}_{i} ) {Φ}_{s} ( {c}_{i} , m )\end{matrix} ] - {α}_{s} / {σ}^{2} \qquad Equation 7$$

In embodiments, one advantage of estimation techniques of this sort is that arbitrary, overlapping feature sets may be used in Φ. For example, trigram, bigram and unigram word features may all be included in the model, as may trigram, bigram and unigram character features, and mixtures of word and character features. Additionally, features may indicate whether a particular word or phrase has occurred previously in the current message, or in the message to which the subject is responding. Topical clusters may be learned, and indicator functions regarding the presence of other words in the topical cluster may be included. Because there is a single, fixed context, the computational overhead at time of inference is far lower than in full sequence prediction tasks such as ASR or MT.

Above, it is noted that the next symbol n may range over a vocabulary Σ that may represent distinct words or phrases. However, in embodiments, sub-word sequences may be presented, such as a single letter. To calculate the probability of a sub-word sequence given the context, one may marginalize (e.g., sum the probabilities) over all symbols that share the prefix of that sub-word sequence. A prefix sub-sequence represents the set of symbols that begin with that prefix. For a prefix p, nεp if the symbol n begins with the subsequence p. Then

P(p|c)=Σ_{nεp}P(n|c) Equation 8

If we present one letter at a time, the first letter in a new word may be a prefix, and the second letter may be an extension to that prefix. For example, if q can be a one-letter extension to the current prefix p, then the conditional probability of q may be calculated as:

$$P ( q \vert c ) = \frac{1}{P ( p \vert c )} {\sum }_{n ∈ pq} P ( n \vert c ) \qquad Equation 9$$

According to embodiments of the disclosed technology, these formulae may provide the means to compare the likelihood of sequences of various lengths for presentation to the subject, and provide non-zero conditional probability to every text symbol, resulting in an open vocabulary system. In an embodiment, in order to obtain a truly open vocabulary, unobserved prefixes may be given non-zero probability by the model. To accomplish this, one-character extensions (e.g., all possible one-character extensions) to the current prefix may be dynamically added to the vocabulary at each step, thus providing at least one vocabulary entry to enable extension of the current prefix. In an embodiment, these dynamic extensions to the vocabulary may not persist past the step in which they were proposed. In an embodiment, model adaptation techniques may guarantee that novel words may eventually be added to the model. In addition to letters, the subject may also be presented with special symbols, including a word-space character such as □; a back-space character such as ←; and/or punctuation characters. In an embodiment, probabilities may be computed several steps in advance of the actual user input and held in reserve for disambiguation by the user. Given expected throughput times, these techniques result in very short latency of symbol prediction.

Embodiments of the disclosed technology also provide methods for presenting stimuli to the user for rapid serial presentation input. In an embodiment, one approach is to present stimuli (e.g., linguistic, nonlinguistic, visual, audible, and the sort) in order of decreasing likelihood (or any other possible order of interest). One possible issue with this approach is potential ambiguity in the signal from the user, so that rapid presentation of many likely stimuli may result in time-consuming errors. Thus, while likelihood of stimuli may remain the main factor determining the sequencing of stimuli in the presentation, other approaches to improving input speed versus a simple likelihood ordering at constant speed may be provided. One embodiment to improve disambiguation makes the duration of a stimulus' presentation a function of its likelihood. Thus, likely stimuli, which may be presented initially, may have a longer presentation duration. Later, if low likelihood stimuli are being accessed, rapid presentation may allow for the identification of a subset of possible stimuli, which may then be re-presented at a longer duration for subsequent disambiguation.

In an embodiment, a key consideration in sequencing of letters may be confusability of letters. Thus letters like b and d may be easily confusable in a rapid presentation, hence, in an embodiment, are not presented adjacently, even if they fall in neighboring ranks in terms of likelihood. Permuting the likelihood ranked list to separate letters with similar shapes may improve throughput by increasing the contrast between letters. Additionally, in an embodiment, briefly masking the site of letter presentation may aid discriminability. Such a technique (separating likely stimuli with less likely stimuli) may be used to distinguish other stimuli from each other.

Of particular utility for contrast may be the special characters mentioned above, such as the backspace character ←, in part because it may be beneficial to provide the user with the ability to revise the input without having to wait for many letters to be presented, even if the probability of revision may be low. For that reason, in an embodiment, presenting such a symbol early in the sequence may be a well-motivated choice for improving discrimination with letters. In embodiments, other “meta-characters” representing editing commands may also have high utility in this capacity.

Finally, in an embodiment, the probability of an extended sequence of letters (e.g., a sequence completing a particularly likely word) may reach a threshold warranting a presentation in entirety, rather than requiring the subject to input each letter in sequence. For example, if the user has input “Manha”, it may be appropriate for the system to suggest “ttan” as the continuation to complete the proper noun “Manhattan.”

In an embodiment, the system may learn from the user's use tendencies. One great benefit of this application is the automatic generation of supervised training data. If the user does not edit what has been generated by the system, an assumption may be that what has been input is correct. This provides on-going updated training data for further improving the model. Such updating is common in certain language modeling tasks, where the domain changes over time. An example of this is automated speech recognition (ASR) of broadcast news, where frequently discussed topics change on a weekly basis: this week is one political scandal; next week is another. Temporal adaptation begins with a particular model, and trains a new model to incorporate (and heavily weight) recently generated data from, for example, newswires. Avoidance of retraining on the entire data set may be a key consideration, for efficiency purposes. Further, in an embodiment, recently collected data may be assumed to be more relevant, to enable models to be specified to a particular user.

In an embodiment, relatively straightforward model adaptation techniques may be utilized. For a small corpus of newly collected sequences, a new log linear model may be trained, using regularized conditional log likelihood as the objective, as presented earlier. In the adaptation scenario, however, one may begin with the current model parameters, and regularize with a Gaussian prior with means at the current parameter values and variances that penalize moving the parameters away from their previous values. In such a way, for example, if there is no data that impacts a particular parameter in the data set, that parameter does not change. If, however, for example, there is a strong likelihood of benefit in the novel data to moving a parameter in one way or the other, the resulting model captures this. A key consideration in this approach may be how frequently to update the model: too frequently may result in sparse data that may cause poor parameter estimation; too infrequently may reduce the impact of adaptation. Similarly, n question seeking empirical validation may be how quickly to move away from the baseline model when new data becomes available from the user (i.e., equivalently, what variance to use in the regularizer). Additionally, akin to model adaptation, but distinct, is the consideration of contextual sensitivity of the models. Sensitivity to contextual factors such as topic or recently used vocabulary may be achieved through features in the log linear model, as described earlier.

A system in accordance with an embodiment of the disclosed technology may utilize the Biosemi ActiveTwo® to collect 32-channel EEG measurements, BCI2000 (a research software toolbox that facilitates real-time interface between standard EEG recording equipment and standard computing languages such including as Matlab® and C) to perform real-time EEG processing, run the natural language models, and present the text sequence on the user's screen. Other suitable known or later developed devices and/or methods may be used for embodiments of the disclosed technology.

In embodiments, various components may be integrated in a real-time closed-loop rapid serial presentation device. FIG. 3 illustrates a closed-loop interface system in accordance with an embodiment of the disclosed technology. As shown in FIG. 3, the system is designed to allow for updates to be driven by other operations of the system to further enhance the functionality of the system.

Thus, embodiments of the disclosed technology provide an innovative brain computer interface that unifies rapid serial presentation, exploits the superior target detection capabilities of humans, noninvasive EEG-based brain interface design capabilities using advanced statistical signal processing and pattern recognition techniques, and intelligent completion ability based on state-of-the-art adaptive sequence models.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope of the disclosed technology. Those with skill in the art will readily appreciate that embodiments in accordance with the disclosed technology may be implemented in a very wide variety of ways. This application is intended to cover any adaptations or variations of the embodiments discussed herein. Therefore, it is manifestly intended that embodiments in accordance with the disclosed technology be limited only by the claims and the equivalents thereof.

What is claimed is:

1. A method, comprising:
presenting at least one sequence of a plurality of stimuli to a user; and
measuring a time course of at least one measurable response to the plurality of stimuli by monitoring at least one input signal of the user.

2. The method of claim 1, wherein monitoring at least one input signal comprises monitoring at least one of an electroencephalogram, an electromyogram, and an evoked response of the user, wherein the response of the user comprises at least one of a neural, physiological, and behavioral response.

3. The method of claim 1, wherein monitoring at least one input signal comprises detecting a positive recognition input signal distinguished from one or more negative input signals.

4. The method of claim 1, wherein monitoring at least one input signal comprises monitoring at least one input signal to correlate the at least one input signal to a nonbinary degree of similarity of the at least one sequence of a plurality of stimuli, wherein the nonbinary degree of similarity comprises at least one of a degree of recognition and a degree of acceptability.

5. The method of claim 1, wherein presenting at least one sequence of a plurality of stimuli comprises presenting a first stimulus followed by a second stimulus, wherein the second stimulus is selected for presentation according to a probability determination that the second stimulus is a next desired element in a sequence desired by the user.

6. The method of claim 5, wherein the probability determination is provided according to a natural language model.

7. The method of claim 6, wherein the natural language model is an adaptive natural language model using input from the user to modify the probability determination.

8. The method of claim 5, wherein the probability determination is provided according to a sequence model.

9. The method of claim 1, further comprising monitoring an attention level of the user.

10. The method of claim 1, wherein presenting at least one sequence of a plurality of stimuli comprises presenting a sequence of linguistic components.

11. The method of claim 1, wherein presenting at least one sequence of a plurality of stimuli comprises presenting a sequence of letters or words.

12. The method of claim 1, wherein presenting at least one sequence of a plurality of stimuli comprises presenting a sequence of nonlinguistic components.

13. The method of claim 1, wherein presenting at least one sequence of a plurality of stimuli comprises presenting a sequence visually.

14. The method of claim 1, wherein presenting at least one sequence of a plurality of stimuli comprises presenting a sequence audibly.

15. A communication system, comprising:
rapid serial presentation of stimuli to a user;
a user intent detection mechanism that employs one or more user signals to indicate intent of the user as to recognition of at least one of the presented stimuli, wherein the one or more user signals comprise at least one of an electrophysiological signal, a neural signal, a behavioral signal, a physiological signal, and a user input signal, and wherein an electrophysiological signal comprises at least one of a multichannel electroencelography (EEG), electromyogram (EMG), and evoked-response potential (ERP); and
a sequence model to predict at least one upcoming stimulus that the user intends so as to control an upcoming sequence of presentation of further stimuli.

16. The system of claim 15, wherein the stimuli are presented visually on a screen at a single position.

17. The system of claim 15, wherein the stimuli comprise linguistic components.

18. The system of claim 15, wherein the stimuli comprise nonlinguistic components.

19. The system of claim 15, wherein rapid serial presentation comprises rapid serial visual presentation.

20. The system of claim 15, wherein rapid serial presentation comprises rapid serial audible presentation.

21. The system of claim 15, wherein the user intent detection mechanism indicates a binary intent of the user as to recognition of at least one of the presented stimuli separate from at least one other of the presented stimuli.

22. The system of claim 15, wherein the user intent detection mechanism indicates a nonbinary degree of intent for recognition or acceptability of at least one of the presented stimuli.
